(19) United States
(12) Patent Application Publication
(10) Pub. No.: US 2022/0354420 A1
(43) Pub. Date: Nov. 10, 2022

(54) METHOD AND SYSTEM USING ACOUSTIC INFORMATION OBTAINED FROM A JOINT AS AN INDICATOR OF A JOINT STATE

(71) Applicant: NIHON UNIVERSITY, Tokyo (JP)

(72) Inventor: Mitsuo NAGAO, Tokyo (JP)

(21) Appl. No.: 17/276,153

(22) PCT Filed: Oct. 30, 2020
(86) PCT No.: PCT/JP2020/040863

§ 371 Date: Mar. 14, 2021

(51) Int. Cl. A61B 5/00; H04R 1/46; A61B 7/00; A61B 5/107

(52) U.S. Cl. CPC A61B 5/4528; H04R 1/46; A61B 7/006; A61B 5/1071; H04R 17/02

(57) ABSTRACT

The problem to be solved is to provide a new method and system for appropriately detecting a joint state from an acoustic of a joint. The present method comprises the step of obtaining, using a bio-acoustic sensor, an acoustic signal emitted by a joint during a time period that at least includes a first motion period wherein the joint changes from a first state to a second state, a pause period of the joint and a second motion period wherein the joint changes from the second state to the first state, the step of converting the obtained acoustic signal into time trend data that at least shows a relationship between an acoustic signal intensity and time, and the step of setting, from the time trend data, a basic threshold value regarding the acoustic signal intensity to calculate first acoustic information based on the basic threshold value, characterized in that the first acoustic information is an indicator of the joint state.

### TECHNICAL FIELD

The present invention is related to a method and system using acoustic information obtained from a joint as an indicator of a joint state.

### BACKGROUND ART

Along with the advancement of aging, patients complaining joint pain is increasing. For example, in the report in the 2008 52nd Annual General Assembly and Scientific Meeting of the Japan College of Rheumatology, according to a wide range of epidemiological investigation, the prevalence rate of knee osteoarthritis from findings of deformation on X-rays is 44.6% for men at the age of 65 or higher and 66.0% for women at the age of 65 or higher, which are high prevalence rates. Since recovery from knee osteoarthritis is very difficult, it is one of the symptoms of which early discovery is especially desirable among joint conditions. Detection of an abnormal sound in a knee joint is examined as a potential method for early discovery.

Upon joint movement, sliding resistance due to degeneration, abrasion, or the like of a cartilage of a joint surface is caused, making a joint sound. This joint sound can be obtained by a bio-acoustic sensor and be utilized as an acoustic signal in diagnosis of arthrosis. Patent Literature 1 discloses an arthrosis diagnosis system using a bio-acoustic sensor, an angle sensor and a weighing meter. In this diagnosis system, the bio-acoustic sensor is attached on a skin near a joint and detects an abnormal sound caused upon bending/stretching of the joint, the angle sensor is attached near the joint and detects a bending/stretching angle of the joint, and the weighing meter detects a load working on the joint as the joint is bent/stretched. In addition, appropriate diagnosis of arthrosis is enabled by distinguishing the abnormal sound obtained by the bio-acoustic sensor from other noises while referring to detection results of the angle sensor and the weighing meter.

### CITATION LIST

### Patent Literature

[PTL 1] Patent Literature 1: Japanese Patent No. 5754689 specification

### SUMMARY OF INVENTION

### Technical Problem

There is a need for a new method and system of appropriately detecting a joint state from an acoustic of a joint using a system with a simple configuration.

### Solution to Problem

In one aspect, the present invention provides a method of using acoustic information obtained from a joint as an indicator of a joint state. The method comprises: the step of, using a bio-acoustic sensor, obtaining an acoustic signal emitted by the joint during a time period at least including a first motion period in which the joint changes from a first form to a second form, a pause period of the joint and a second motion period in which the joint changes from the second form to the first form; the step of converting the acoustic signal that was obtained to time trend data at least showing a relationship between an acoustic signal intensity and time; and the step of setting a basic threshold value regarding the acoustic signal intensity from the time trend data and calculating first acoustic information based on the basic threshold value, characterized in that the first acoustic information is used as an indicator of the joint state.

In one embodiment of the method of the present invention, setting a basic threshold value regarding the acoustic signal intensity from the time trend data further comprises identifying an initial time and a terminal time of at least one non-signal time period from the time trend data and calculating the basic threshold value from time trend data in the non-signal time period. At least one of the initial time and the terminal time of the non-signal time period is identified under the condition of an absolute value of an average change rate of the acoustic signal intensity in a predetermined time period being a predetermined value or lower. The basic threshold value is calculated based on an average value and a standard deviation of an acoustic signal intensity in the non-signal time period in the time trend data and a first coefficient.

In one embodiment of the method of the present invention, calculating the first acoustic information based on the basic threshold value comprise calculating the first acoustic information by extracting an acoustic signal intensity exceeding the basic threshold value in the time trend data.

In one embodiment of the method of the present invention, converting the acoustic signal that was obtained to the time trend data at least showing a relationship between the acoustic signal intensity and time comprises digitally converting the acoustic signal that was obtained on a predetermined sampling frequency. The predetermined sampling frequency is about 25 Hz to about 2000 Hz. The first acoustic information is a count and/or intensity of the digitally converted acoustic signal exceeding the basic threshold value.

In one embodiment of the method of the present invention, the first acoustic information is calculated from an acoustic intensity corresponding to the first motion period in the time trend data.

In one embodiment of the method of the present invention, comparison between the first acoustic information and a pre-set discrimination threshold value is used as an indicator of the joint state.

In one embodiment of the method of the present invention, the step of calculating the acoustic information further comprises setting an additional threshold value of an acoustic signal intensity that is higher than the basic threshold value and calculating second acoustic information, wherein a combination of the first acoustic signal and the second acoustic signal is used as an indicator of the joint state.

In one embodiment of the method of the present invention, comparison between a combination of the first acoustic information and the second acoustic information and a pre-set discrimination value is used as an indicator of the joint state.

The joint to which the method of the present invention is applied may be a knee joint or an elbow joint.

In another aspect, the present invention also provides a system of detecting a joint state in accordance with the present invention. The system comprises: a bio-acoustic sensor obtaining acoustic information from a joint; and a detection apparatus detecting a joint state from a result of the acoustic information obtained by the bio-acoustic sensor.

In one embodiment of the system of the present invention, the system comprises: a data conversion part converting the acoustic signal obtained with the bio-acoustic sensor to time trend data at least showing a relationship between an acoustic signal intensity and time; a threshold setting part setting a basic threshold value regarding the acoustic signal intensity from the time trend data; an acoustic information calculation part calculating first acoustic information based on the basic threshold value; and an indicator calculation part calculating an indicator of a joint state based on the first acoustic information that was calculated.

In one embodiment of the system of the present invention, the system does not comprise an angle sensor.

### Advantageous Effects of Invention

The present invention provides a new method of estimating a joint state from an acoustic obtained with a bio-acoustic sensor and a system therefor. Since this enables a subject to easily measure the joint state, it is expected that this would contribute to monitoring of the joint state of the subject or early discovery of an abnormality.

### BRIEF DESCRIPTION OF DRAWINGS

FIG. 1 is a flowchart of the method of the present invention using acoustic information obtained from a joint as an indicator of a joint state.

FIG. 2 is a front view of a configuration of a knee joint 200 of a human.

FIG. 3 shows time trend data of an acoustic signal obtained from a bio-acoustic sensor with an analog signal and a digital signal at a spectrum level.

FIG. 4 is a data processing graph 400 showing a method of setting a basic threshold value.

FIG. 5 shows an example of a method of detecting a progression phase of knee osteoarthritis from first acoustic information calculated using the basic threshold value 454 in FIG. 4.

FIG. 6 shows data associated with first acoustic information.

FIG. 7 is a flowchart of the method of the present invention using first and second acoustic information obtained from a joint using a basic threshold value and an additional threshold value as indicators of a joint state.

FIG. 8 is a data processing graph 800 showing a method of setting a basic threshold value and an additional threshold value.

FIG. 9 is a flowchart of the method of the present invention using first, second, third and fourth acoustic information obtained from a joint as an indicator of a joint state.

FIG. 10 is a data processing graph 1000 showing a method of setting a basic threshold value, first additional threshold value, a second additional threshold value and a third additional threshold value.

FIG. 11 shows an example 1100 of a joint state detection system.

FIG. 12 shows a structure 1200 of an elbow joint.

### DESCRIPTION OF EMBODIMENTS

The present example is described below. Unless specifically referred to, the terms used herein should be understood as being used in the meanings that are generally employed in the subject field. Therefore, unless defined otherwise, all of the technical terms and scientific terms used herein have the same meaning as those generally understood by those skilled in the art in the field to which the present invention belongs. In a case of contradiction, the present specification (including definitions) will be prioritized.

Herein, “about” refers to the concept of being within the range of ±10% of the number thereafter.

The present invention is related to a new method and system using acoustic information obtained from a joint as an indicator of a joint state. Embodiments of the present invention are described below in detail using the drawings. First, a method using acoustic information obtained from a joint as an indicator of a joint state by using a basic threshold value is described as a first embodiment.

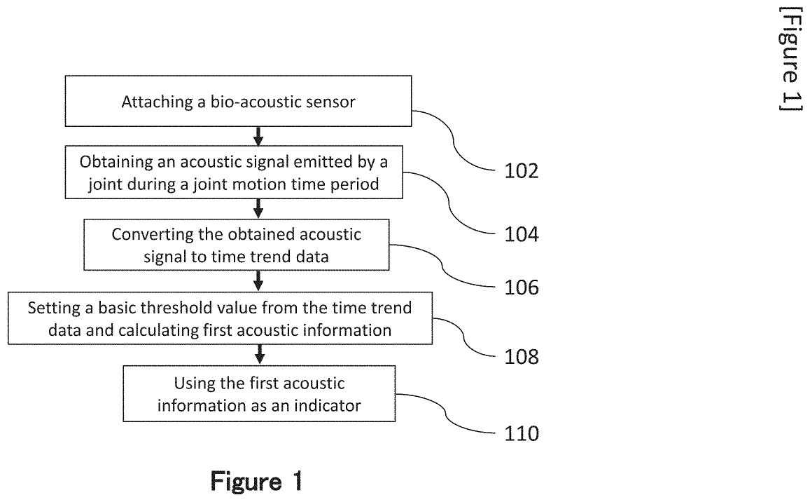

FIG. 1 is a flowchart of the method of the present invention using acoustic information obtained from a joint as an indicator of a joint state. First, in step 102, a bio-acoustic sensor is attached on a skin surface near a joint of a measuring object of a subject. The bio-acoustic sensor may be in any form as long as it is possible to detect a biological sound of a human. For example, the bio-acoustic sensor is a type of microphone that detects a biological sound of a human, comprising a sensor device that detects vibration caused by the biological sound. Various forms of sensor devices such as a voice coil type microphone, a capacitor microphone, or a piezoelectric element can be used. Preferably, the sensor device is a piezoelectric element that can detect a very small sound. When using a piezoelectric element as the sensor device, a skin vibrates due to a joint sound caused upon moving the joint, the piezoelectric element stretches/shrinks due to the vibration, and this stretching/shrinking is detected as a piezoelectric signal. The detected piezoelectric signal is obtained as an acoustic signal expressing the joint sound. In addition, it is preferable that the bio-acoustic sensor has a structure of suppressing other noises, especially a noise sound caused by movement of an organism, from entering upon detecting a joint sound. In one embodiment, the bio-acoustic sensor may suppress a noise using a semiconductor circuit and an earth wire for noise removal. In another different embodiment, the bio-acoustic sensor may suppress a noise by mitigating vibration added to a sensor device from outside by covering a member surrounding the piezoelectric element with a buffer.

The method in the present invention may be applied to detection of a joint state in various joints. For example, joints that may be targeted by the present invention include, but not limited to, a knee joint, an elbow joint, a finger joint, a shoulder joint, a wrist joint, an ankle joint, a hip joint, a jaw joint and so on. In a preferable embodiment, the present invention may be used in a knee joint, an elbow joint and a jaw joint, which generally repeatedly move mainly between a first form and a second form. More preferably, the present invention may be used in a knee joint and an elbow joint with more tendency to show joint state abnormality as acoustic information compared to other joints. Especially preferably, the present invention may be used in a knee joint.

In addition, the bio-acoustic sensor in the present invention may be invasive, or may be non-invasive. Preferably, the bio-acoustic sensor in the present invention is a non-invasive sensor that can easily be worn near a joint that is to be a measuring object. The means for wearing a non-invasive sensor near a joint may be any means. For example, the non-invasive sensor may be attached on a skin with an adhesive tape, or may be fixed on a skin near a joint of a subject with an appropriate band tool.

The bio-acoustic sensor is at least one or more for a joint of which joint state is sought to be detected, wherein any number of bio-acoustic sensors may be worn. For example, when a more accurate state of a joint is sought to be detected or the like, bio-acoustic sensors may be, for example, provided at a plurality of sites at the upper part, the inside and the outside of the joint to detect a difference or the like in the joint state between the inside portion and the outside portion.

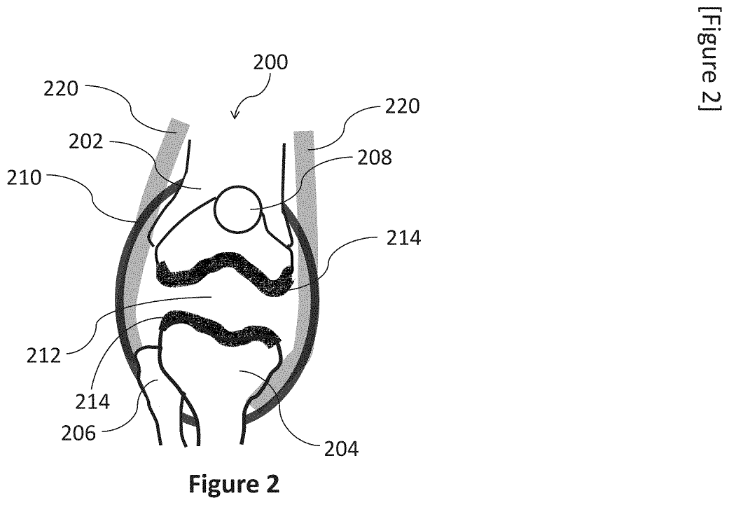

While the location near a joint to which a bio-acoustic sensor is attached may be any location as long as it is possible to sense a joint noise in the joint, it is preferable that a bio-acoustic sensor is at a position that is as close as possible to the location where a joint sound is generated. FIG. 2 is a front view of a configuration of a knee joint 200 of a human which is a representative target of application of the present invention. A knee joint mainly consists of a femur 202, a tibia 204, a fibula 206 and a patella 208 as well as many layers of muscles and ligaments 220 existing while wrapping around the above. A knee joint is wrapped with a joint capsule 210. Cartilages 214 are attached to the joint surface where the femur 202 and the tibia 204 are facing each other, wherein the joint surface is filled with synovial fluid 212 that plays a role as a lubricating oil. The cartilages 214 on the joint surface slide with respect to one another when the knee joint is bent, which sliding generates a joint noise. An abnormality of a joint such as knee osteoarthritis may especially appear in a joint noise caused by the sliding of cartilages 214 on the joint surface with respect to one another.

Therefore, in one embodiment, when a bio-acoustic sensor is provided near a knee joint, it is desirable that the bio-acoustic sensor is attached near a lower part of the patella 208 where the joint surface exists. In a preferable embodiment, the present invention can, for example, detect a joint sound of the joint surface where the femur 202 and the tibia 204 face each other with one acoustic sensor and use the joint sound as an indicator of a joint state. In another embodiment, in addition to an acoustic of the joint surface where the femur 202 and the tibia 204 face each other that is detected by one acoustic sensor, it is also possible to detect an acoustic inside and/or outside a joint with another one or two acoustic sensor and combine these to collectively estimate a joint state with good precision using more information.

In step 104, in a state in which a bio-acoustic sensor is worn, the subject moves the joint from a first form to a second form. In the repeating motion between the first form and the second form, this time period for moving the joint from the first form to the second form is called a “first motion period” herein. For example, in a knee joint, the first form may be a bent form in which the knee joint is closed and the second form may be an extended form in which the knee joint is open, or the first form may be an extended form in which the knee joint is open and the second form may be a bent form in which the knee joint is closed. In addition, after maintaining the second form for a predetermined time period (herein, this time period is called a “pause period” of the joint), the joint is moved so as to return from the second form to the first form. This time period for moving the joint from the second form to the first form is called a “second motion period” herein. A bio-acoustic sensor obtains an acoustic signal emitted by the joint during the time from the start of this first motion period, through the pause period, to the end of the second motion period (herein, this is called a “joint motion time period for one reciprocation”). It is possible to cover all forms and motions of the joint that are necessary for the present invention to estimate the joint state by obtaining an acoustic signal (joint sound) in a joint motion during a joint motion time period of one reciprocation with a bio-acoustic sensor. A joint sound in a joint motion time period of a plurality of reciprocations may be obtained in order to increase detection precision.

In addition, the magnitude of the load applied to a knee joint affects the magnitude of a joint sound. Thus, a bending/stretching motion may carry out both an active bending/stretching which is carried out while remaining in a sitting position without the foot touching the floor and a loaded bending/stretching which is a shift from a sitting position to a standing position to obtain an acoustic signal in each bending/stretching motion. Both the active bending/stretching and the loaded bending/stretching do not necessarily need to be performed. Only one may be performed. Compared to the active bending/stretching, the loaded bending/stretching has a great load applied to the knee joint, thereby generating a large joint sound. In this regard, it is better to carry out the loaded bending/stretching, in which a stronger acoustic signal can be obtained, for easier detection of the joint state.

In step 106, acoustic signals obtained by a bio-acoustic sensor are converted to time trend data by the detection apparatus discussed below. The time trend data is data in which the obtained acoustic signals are aligned in chronological order, at least showing the relationship between acoustic signal intensity and time.

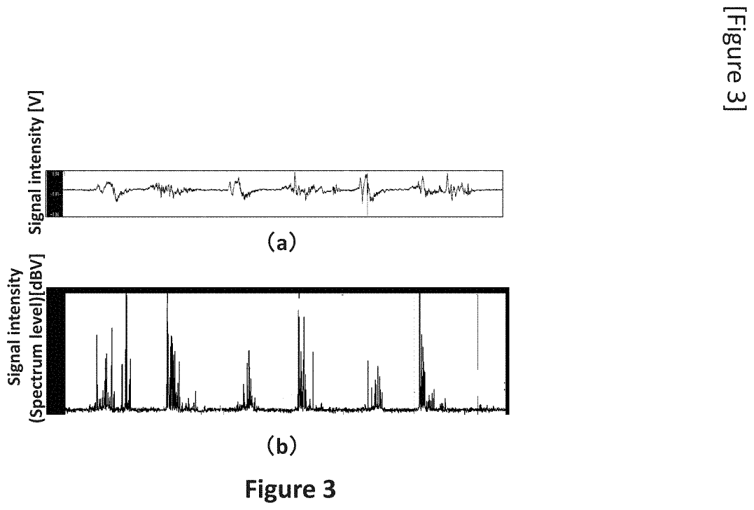

FIG. 3 shows one example of time trend data, wherein FIG. 3(a) is a figure wherein analog electric signals obtained from a bio-acoustic sensor are aligned in chronological order. FIG. 3(b) shows data wherein the time trend data of FIG. 3(a) has been converted to digital signals at a spectrum level. The detection apparatus preferably may convert this analog electric signal to a digital signal on a predetermined sampling frequency by analog-digital (AD) conversion, in order to turn an acoustic signal into a numerical value. The AD conversion can carry out conversion by any means known in the subject field, which can be performed by, for example, a hardware such as an AD conversion apparatus or a software such as an application. The bio-acoustic sensor is connected to a hardware or software of a detection apparatus by wire communication or wireless communication. When AD conversion is used, it is desirable that a sampling frequency is set to a constant value in order to the first acoustic information to be calculated as a count of signals and be usable as an indicator. However, in a case of comparison with acoustic information of other sampling frequencies, even with an indicator calculated on a different frequency, the count of acoustic signals may be modified by multiplying the ratio of the sampling frequency on which this indicator has been obtained and the sampling frequency of the comparison target. While a predetermined sampling frequency is selected from between about 25 Hz to about 2000 Hz, it is preferable to secure data of the count of acoustic signals used as acoustic information of a certain degree in order to appropriately evaluate the joint state, whereas it is better that the sampling frequency is not too great for simply carrying out data processing in a short time. Therefore, the sampling frequency in the present invention is about 30 Hz to about 500 Hz, more preferably about 50 Hz to about 300 Hz, and most preferably about 100 Hz. The signal intensity shown in FIG. 3(b) may be, for example, at a spectrum level obtained by processing an acoustic signal as a short-time Fourier transform processing signal, which is expressed with a dB (decibel) unit.

In step 108, the detection apparatus sets a basic threshold value from the time trend data to calculate the first acoustic information. The basic threshold value is the acoustic signal intensity that is used as a criterion upon calculating the acoustic information that is used as an indicator of the joint state from an acoustic signal. The basic threshold value may be set to a predetermined acoustic signal intensity, or may be set to a value calculated from an acoustic signal obtained from each subject. Since the acoustic signal intensity upon moving a joint that is obtained by a bio-acoustic sensor differs among individuals, it is preferable to calculate a basic threshold value from an acoustic signal obtained from each subject. This enables suppression of variation in the difference in measured values among individuals and enables increase of precision of detection of a joint state.

In one embodiment, when a predetermined acoustic signal intensity is used as a basic threshold value, the acoustic signal intensity is set based on the measurement results obtained from various subjects.

When calculating a basic threshold value for each subject, the basic threshold value, for example, may be set to an average value of acoustic signal intensity obtained throughout the entire time period of measurement, or may be set to a value calculated from the acoustic signal intensity in a time period in which signals with low acoustic signal intensity are in succession. Herein, the time period in which signals with an acoustic signal intensity lower than a predetermined condition are in succession is called a “non-signal time period”. Since it can be considered that an acoustic signal with high acoustic intensity locally existing in time trend data expresses a joint sound due to sliding of bones with respect to one another upon joint motion, it is preferable for acoustic information to be calculated from the acoustic signal intensity excluding a signal during a non-signal time period in order to detect a joint state with good precision. For example, when bending/stretching motion of a joint is not carried out or the like, the acoustic signal intensity should originally be at zero value from a bio-acoustic sensor, but a signal including various noises and with low acoustic signal intensity is actually generated.

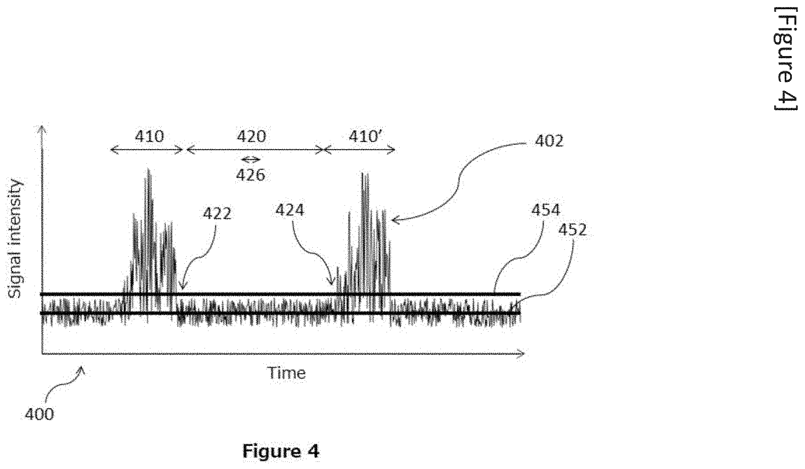

FIG. 4 is a figure explaining a method of setting a basic threshold value, showing a data processing graph 400. The time trend data 402 in the graph 400 is data wherein acoustic signals obtained from a bio-acoustic sensor throughout a time period including at least a joint's first motion period, pause period and second motion period are aligned in chronological order and converted to digital signals at a spectrum level in the same manner as FIG. 3(b). The vertical axis shows acoustic signal intensity and the horizontal axis shows time. The time trend data 402 consists of acoustic signal emission time periods 410 and 410′ in which signals with high acoustic signal intensity are focused and a non-signal time period 420 in which signals with low intensity are in succession. In a case of a bending/stretching motion of a joint, while it is considered that the acoustic signal emission time periods 410 and 410′ correspond approximately to the first motion period and the second motion period and the non-signal time period 420 corresponds approximately to the pause period in which the bending/stretching motion is paused, it should be noted that the present invention does not necessarily require accurate identification of a motion period and a pause period. For example, it is possible to estimate a joint state by calculating the acoustic information that is used as an indicator of the joint state only from an acoustic signal, independently from the bending/stretching motion of the joint, wherein while it is preferable that the acoustic signal emission time period 410 and the time period in which the bending/stretching motion of the joint was actually carried out match, they do not necessarily need to match.

In one embodiment, the acoustic signal intensity of a non-signal is first identified in order to calculate the basic threshold value 454. The acoustic signal intensity of a non-signal may be calculated using the acoustic signal intensity in the non-signal time period 420 as a criterion. The non-signal time period 420, for example, may be determined by identifying an initial time 422 and a terminal time 424 thereof, or may be identified as a time period below a value of a signal intensity that was predetermined. Since the variation in acoustic signal intensity in the non-signal time period 420 differs among individuals, it is preferable to identify the initial time 422 and the terminal time 424 of the non-signal time period 426 for each subject.

In a representative embodiment, the initial time 422 and the terminal time 424 of the non-signal time period 420 may be identified under the condition of the absolute value of an average change rate of the acoustic signal intensity for each micro time being a predetermined value or lower successively throughout a predetermined time period 426. A micro time is a time that is sufficiently short compared to the non-signal time period 420 and the acoustic signal emission time periods 410 and 410′, which is preferably about 0.04 seconds or shorter. When an acoustic signal is digitally converted, a micro time may be a sampling cycle, and when a sampling frequency is 100 Hz, a micro time may be 0.01 second. In addition, the predetermined time period 426 may be any time period that can distinguish the non-signal time period 420 from the acoustic signal emission time periods 410 and 410′, and includes a plurality of the aforementioned micro times. For example, when a micro time is about 0.01 second, the predetermined time period 426 may be about 0.2 seconds.

When the absolute value of the average change rate of the acoustic signal intensity of a micro time is at or below a predetermined value successively throughout the predetermined time period 426, the initial time of the predetermined time period 426 may be identified as the initial time of the non-signal time period 420. In addition, when the absolute value of the average change rate of the acoustic signal intensity of a micro time is over the predetermined value in the predetermined time period 426, the terminal time of the predetermined time period 426 may be identified as the terminal time of the non-signal time period 420. The predetermined value may be appropriately adjusted in accordance with the length and acoustic signal intensity (unit) of the micro time.

As shown in FIG. 4, in the time trend data 402, first, since signals with high acoustic signal intensity and signals with low acoustic signal intensity are mixed in the acoustic signal emission time period 410, the absolute value of the average change rate of the acoustic signal intensity of a micro time would be great and the value thereof would greatly vary. Then, the acoustic signal intensity quickly decreases and acoustic signals with small acoustic signal intensity would appear successively. At this time, the absolute value of the average change rate gradually decreases, and when signals with small acoustic signal intensity starts to appear successively, the absolute value of the average change rate would be a value that is close to 0. Therefore, the time point when the absolute value of the average change rate is below the predetermined value successively throughout the predetermined time period 426 can be determined as the initial time 422 of the non-signal time period 420. Then, the time trend data quickly changes from a state in which signals with low acoustic signal intensity appear successively to signals with high acoustic signal intensity, thereby causing the absolute value of the average change rate of acoustic signal intensity of the micro time to be greater. This greatness can identify the time point when the predetermined value has been exceeded in the predetermined time period 426 as the terminal time 424 of the non-signal time period 420.

A basic threshold value 454 is set based on the acoustic signal intensity of an acoustic signal within the non-signal time period identified by the initial time 422 and the terminal time 424. The basic threshold value 454 may be, for example, the average value of acoustic signal intensity within the non-signal time period 420, a value resulted by adding a constant value to the average value of acoustic signal intensity within the non-signal time period 420, a value calculated from the average value of acoustic signal intensity within the non-signal time period 420 and the variation in signal intensity within the non-signal time period 420, or may be calculated as the maximum intensity within the non-signal time period 420. There is variation in acoustic signal intensity within the non-signal time period 420, and in order to appropriately evaluate this variation and set a basic threshold value 454, it is desirable that the basic threshold value 454 be set to a value calculated from the average value of acoustic signal intensity within the non-signal time period 420 and the variation in acoustic signal intensity within the non-signal time period 420. In this case, the average value of acoustic signal intensity within the identified non-signal time period 420 (average value of non-signal intensity 452 (Ave.)) is calculated, and, additionally, a standard deviation (SD) of acoustic signal intensity within the non-signal time period 420 is calculated as the variation in acoustic signal intensity within the non-signal time period 420. In a typical embodiment, a basic threshold value is calculated with TH=Ave.+αSD (formula 1) and the coefficient α of the standard deviation (SD) can be arbitrarily set. The coefficient α may be set so as to include, 80% or more, more preferably 90% or more, and typically all, of the acoustic signals within the non-signal time period 420. It is preferable that the coefficient α be set so as to not include an acoustic signal of time periods other than the non-signal time period 420 (an acoustic signal that originally should belong to the acoustic signal emission time period 410 or 410′) to the extent possible and not leak an acoustic signal of the non-signal time period 420. For example, a coefficient α is set to a value that is 3 or higher. Empirically, in order to extract as much signals having the intensity that exceeds a signal within the non-signal time period 420 as possible as acoustic information while excluding almost all signals within the non-signal time period 420 from the acoustic information with a basic threshold value, it is more preferable that the coefficient α be set to 3.

The first acoustic information may be calculated from the set basic threshold value 454. The first acoustic information may be in any form as long as calculation from the basic threshold value 454 is possible, which, for example, may be extracted as the acoustic signal intensity that exceeds the basic threshold value 454, may be a ratio of the time in which the acoustic signal intensity exceeds the basic threshold value 454 to the entire measurement time, may be the average value of signal intensity exceeding the basic threshold value 454, or, when the acoustic signal is a digital signal, may be a count of signals having an intensity that exceeds the basic threshold value and/or said intensity.

In the time trend data 402, the first acoustic information may be calculated from i) only a time period corresponding to a first motion period in which a joint changes from a first form to a second form, ii) only a time period corresponding to a second motion period in which a joint changes from a second form to a first form, iii) a time period corresponding to a joint motion period of one reciprocation, iv) a time period corresponding to a joint motion period of a plurality of reciprocations, or the like. Which time period to be selected depends on the desired information. For example, the motion period of changing from a bent form to an extended form and the motion period of changing from an extended form to a bent form actually differ in terms of the sliding joint surface and also differ in terms of the generated acoustic signal. Therefore, in a joint motion, it is preferable to calculate the first acoustic information based on the motion period in which the acoustic signal intensity is expressed significantly. For example, when a subject feels pain in a joint only upon the motion period of changing from an extended form to a bent form, the count of signals of only the motion period of changing from an extended state to a bent state may be calculated to use the count as an indicator for detection of a joint state. In addition, a joint state can be understood with more detail by calculating all of the counts of the signals of each time period of i) to iv).

In step 110, an indicator of a joint state is obtained from the obtained first acoustic information. Since the first acoustic information is a numerical value expressing the signal intensity, count of signals or the like that is calculated with the above-described method, it is possible to detect the joint state with this numerical value. For example, the first acoustic information may be an indicator of knee osteoarthritis, knee osteochondritis dissecans, meniscal damage, or the like. In addition, the first acoustic information may be an indicator showing not only a disease but also a daily joint state. When the first acoustic information is an indicator showing a daily joint state, it is possible to detect whether the joint state on the day of measurement is good or bad by comparison with the first acoustic information of himself/herself calculated in the past or comparison with a standard value of himself/herself. Meanwhile, when detecting a state of a disease such as knee osteoarthritis, it is preferable to compare information calculated as the first acoustic information with objective information.

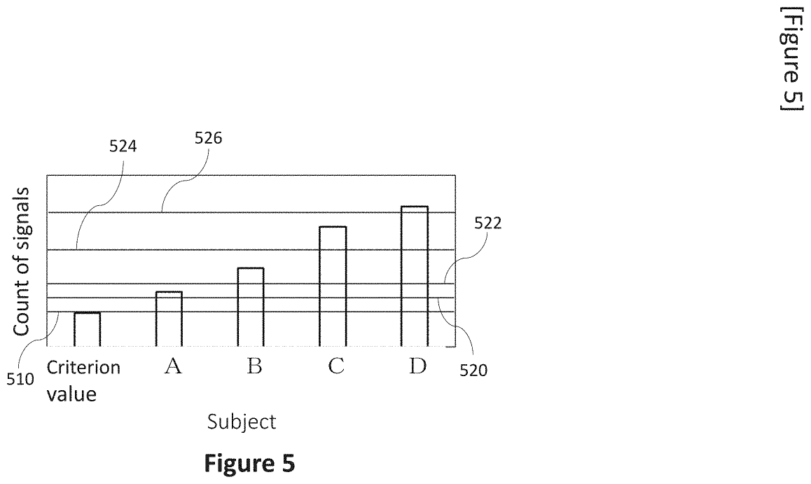

When detecting a state of a disease with the first acoustic information, an indicator showing a joint state may be a comparison between the acoustic information and a pre-set discrimination threshold value. FIG. 5 shows one example of a method of detecting a progression phase of knee osteoarthritis from the first acoustic information calculated using the basic threshold value 454 in FIG. 4. In this example, the first acoustic information is a count of signals exceeding the basic threshold value 454. The discrimination threshold value may be a ratio with respect to a criterion value 510. The criterion value 510 may be a count of signals detected in a healthy knee joint. The criterion value 510 is incorporated in a system (e.g., detection apparatus) beforehand, inputted via a device or the like, or may be set via wire communication or wireless communication and regularly updated. The discrimination threshold value may be associated with, for example, the symptom classification of knee osteoarthritis. There is Kellgren-Lawrence classification (K-L grade) as the symptom classification of knee osteoarthritis. The K-L grade is an indicator used in the medical scene as classification of severity of knee osteoarthritis, wherein a knee state is confirmed and classified based on an X-ray image of a knee joint of a subject. The K-L grade has five stages from grade 0 to grade 4 (G4), wherein grade 0 (G0) is normal, suspicion of joint fissure narrowing or a mild degree of spur formation can be seen in grade 1 (G1), a mild degree of joint fissure narrowing or spur formation can be seen in grade 2 (G2), a plurality of spur formations, joint fissure narrowing, or subchondral sclerosis can be seen in grade 3 (G3), spur formation, advanced degree of joint fissure narrowing, or advanced degree of subchondral sclerosis can be seen in grade 4 (G4). For example, a count of acoustic signals corresponding to G0 is set as the criterion value 510. In addition, a count of signals that is 1.5-fold of the criterion value may be a first discrimination threshold value 520 detected as corresponding to G1, a count of signals that is 2-fold may be a second discrimination threshold value 522 detected as corresponding to G2, a count of signals that is 3-fold may be a third discrimination threshold value 524 detected as corresponding to G3, and a count of signals that is 4-fold may be a fourth discrimination threshold value 526 detected as corresponding to G4. According to the criterion value 510 and first to fourth discrimination threshold values 520, 522, 524 and 526, subject A is detected as G1, subject B is detected as G2, subject C is detected as G3 and subject D is detected as G4.

This detection may also be, for example, two steps of “normal” and “abnormal”, or three stages of “healthy”, “pre-knee arthrosis” and “knee arthrosis”. The discrimination threshold value may be set using accumulated data of many subjects or the like as a criterion.

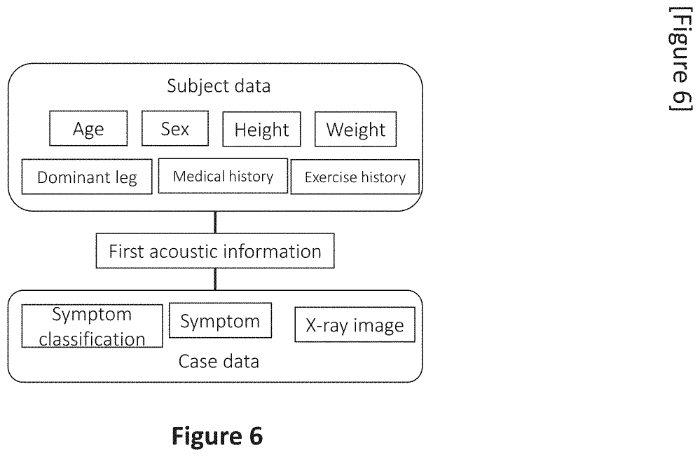

FIG. 6 shows one example of data associated with the first acoustic information. In detection of a joint state, the first acoustic information may be associated with subject data and case data. The subject data may include at least one of a subject's age, sex, height, weight, dominant leg, medical history and exercise history. For example, since aged people generally have fatigued joint surface and large degree of cartilage abrasion compared to young people, it is highly likely that the first acoustic information is detected as having an abnormal joint state. In this regard, the first acoustic information may be associated with age and compared with the criterion value (discrimination threshold value) corresponding to the age. In addition, acoustic signal intensity tends to be strong right after an exercise such as sports, and as a result, an error may occur in the detection of a joint state. Thus, the criterion value (discrimination threshold value) may be corrected to a value corresponding to the exercise history by associating the first acoustic information with the exercise history, or a re-measurement after some time may also be requested. In addition, the case data may include a specific symptom, X-ray image and the above-discussed symptom classification. A case corresponding to the first acoustic information can be searched by accumulating data associating first acoustic information and case data of other subjects. Accordingly, an indicator for detecting a joint state with more precision can be generated by associating the first acoustic information calculated from the method of the present invention with other data.

The present invention can calculate the first acoustic information that is used as an indicator of a joint state using only an acoustic signal obtained from a bio-acoustic sensor as discussed above. Therefore, the present invention is especially useful in terms of not needing to detect the progression/timing of a joint motion of a subject using other sensors such as a conventional angle sensor or a weighing meter to cross-reference with time trend data of an acoustic signal and being able to achieve low cost with a simple apparatus configuration. In addition, a weighing meter is an apparatus in any form that can measure motion acceleration. For example, the acceleration sensor may include a vibration system, an optical system and a semiconductor system. Preferably, the acceleration sensor is a semiconductor system (e.g., piezo-resistance type or electrostatic capacitance type) that can be miniaturized. In addition, the angle sensor is an apparatus in any form that can measure an angle and an angular speed. For example, the angle sensor may be a rotary sensor, inclination sensor, or may be a gyro sensor. Furthermore, while an angle sensor and a weighing meter are basically unnecessary, those apparatuses may be combined and used as needed in the present invention.

A conventional system of diagnosing a joint state from a joint sound not only uses a bio-acoustic sensor but also uses an angle meter and a weighing meter. This conventional system needed to use an angle sensor and a weighing meter to accurately detect the timing of a subject's motion of standing up from a chair for a joint motion and refer to this detection result to remove a noise involved in the motion of the detection data of a bio-acoustic sensor. As such, a conventional diagnosis system needs many sensors, becoming a high-cost apparatus with complicated apparatus configuration, thereby only being able to be practiced in a medical institution or the like. Meanwhile, since early discovery is important in joint diseases such as knee osteoarthritis as discussed above, easy detection of a joint state on a daily basis at home or the like is desired to be able to be carried out.

According to the present invention, it is possible to obtain a method and system for appropriately detecting a joint state from an acoustic of a joint, which can be easily practiced at home or the like using only a bio-acoustic sensor. The method and system of the present invention can naturally be used in medical institutions or the like as well, wherein, for example, a joint state can also be diagnosed in combination with X-ray imaging and examination by a physician.

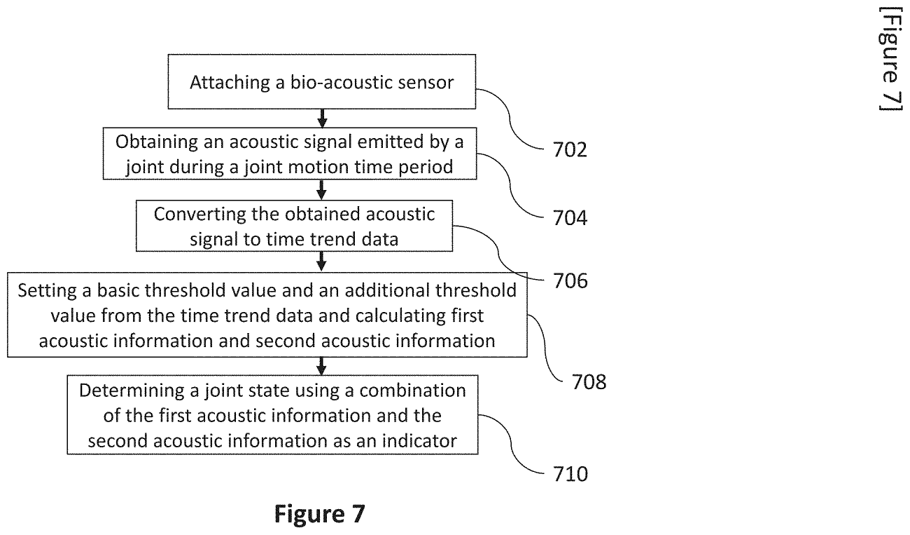

Next, a second embodiment is described. FIG. 7 shows a flowchart of the method of the present invention using first and second acoustic information obtained from a joint using a basic threshold value and an additional threshold value as an indicator of a joint state. The second embodiment is different compared to the first embodiment in terms of calculating second acoustic information aside from the first acoustic information. Since step 702 to step 706 are similar to step 102 to step 106 in the first embodiment, the description thereof is omitted, and step 708 and step 710, which are steps different from the first embodiment, are described.

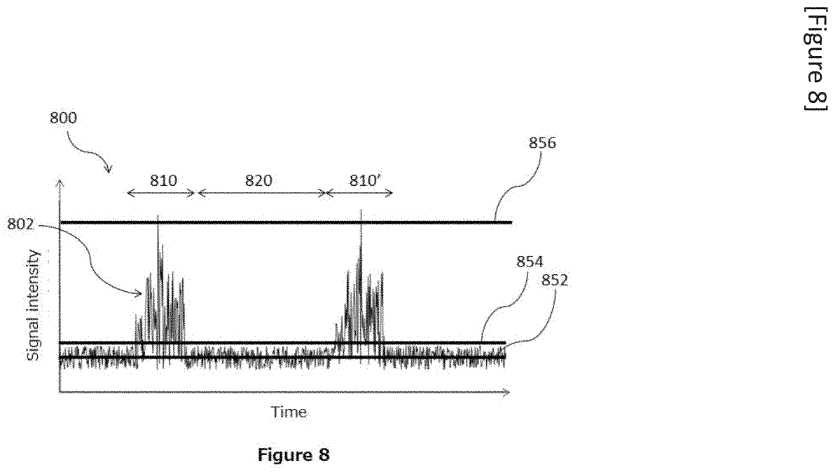

In the present embodiment, in step 708, a basic threshold value and an additional threshold value are set based on the time trend data to calculate the first and second acoustic information. FIG. 8 is a data processing graph 800 showing a method of setting a basic threshold value and an additional threshold value. In the same manner as the case of the graph 400 shown in FIG. 4, the basic threshold value 854 may be set by identifying a non-signal time period 820 from the graph 800 and calculating an average value 852 of non-signal intensity. The present embodiment sets an additional threshold value 856 in addition to the basic threshold value 854.

An additional threshold value 856 may be arbitrarily set as long as the value has the acoustic signal intensity that is different from the basic threshold value 854, which may be a value higher than or a value lower than the basic threshold value 854. Since extraction of an acoustic signal with higher acoustic signal intensity extracted using the additional threshold value as the second acoustic information while using the first acoustic information extracted using the basic threshold value 854 as a basis is effective for generating an indicator of a joint state with higher certainty, it is preferable that the additional threshold value 856 is set to a value higher than the basic threshold value 854.

The set value of the additional threshold value 856 may be any value. For example, the value may be set to a pre-set acoustic signal intensity, or may be set to a value calculated from an acoustic signal obtained from each subject.

The additional threshold value 856 can be calculated in any way. In one embodiment, said value can be calculated with (formula 1) in the same manner as the basic threshold value. The additional threshold value 856 can be calculated by differentiating a value of a coefficient a of a standard deviation (SD) of (formula 1) from a value used to calculate the basic threshold value 854. For example, when set to a signal intensity higher than the basic threshold value 854, an additional threshold value 856 may be a value calculated while replacing a first coefficient in a similar formula with a second coefficient with a higher value. This further eliminates some of the acoustic signals with comparatively high acoustic signal intensity in the signal emission time periods 810 and 810′ in addition to the non-signal time period 820. The first acoustic information may be calculated from the basic threshold value 854 and the second acoustic information may be calculated from the additional threshold value 856. Since an acoustic signal having an intensity exceeding the additional threshold value 856 may present information that is different from the first acoustic signal derived from the basic threshold value, combination of the first and second acoustic information enables detection of a joint state with higher precision. An embodiment using such an additional threshold value 856, for example, regardless of having a small count of signals having the acoustic signal intensity exceeding the basic threshold value 854, can appropriately detect an abnormality in a joint state of a subject with a characteristic of the signals having an extremely great intensity. For example, when a joint has an abnormality such as having a joint surface portion with extremely serious damage in the joint, a sound with a very high acoustic signal intensity is caused, thus enabling detection of the presence/absence of the existence of a joint surface portion with extremely serious damage with the second acoustic information showing the presence/absence, count and the like of the acoustic signals exceeding the additional threshold value 856.

In step 710, a joint state is detected using the combination of the first acoustic information and the second acoustic information as an indicator. The present embodiment may also carry out detection of a joint state using the discrimination threshold value described in FIG. 5. In addition, in the present embodiment, even when, for example, detected as G2 based on the first acoustic information, when there is a signal having an acoustic signal intensity exceeding the additional threshold value 856 based on the second information, it is possible to carry out a more multilateral estimation of a joint state such as increasing the progression phase by one stage and detecting that the joint state is in G3. In addition, detection of a joint state with higher precision may be carried out by weighting each of the count of signals of the first acoustic information and the count of signals of the second acoustic information (e.g., weighting the count of signals of the first acoustic information to about 1-fold and weighting the second acoustic information to about 5-fold). Furthermore, the weighting can be carried out under any condition and adjustment can be appropriately made. In addition, when the second acoustic information detects an abnormally high acoustic signal intensity, re-measurement may be instructed in consideration of the possibility of this value being an error (noise). When the second acoustic information still detects an abnormally high acoustic signal intensity even after re-measurement, this would be handled as the second acoustic information.

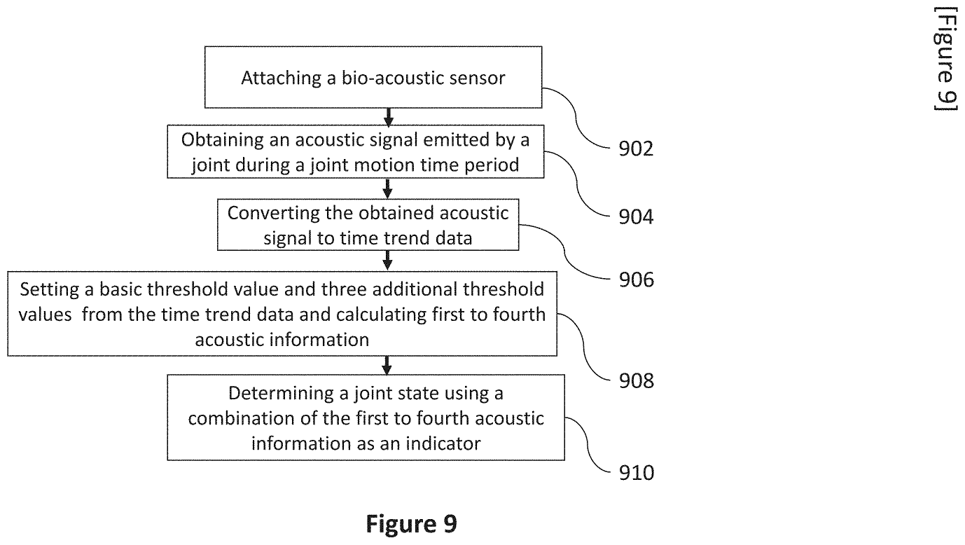

Next, a third embodiment is described. FIG. 9 is a flowchart of the method of the present invention using first, second, third and fourth acoustic information obtained from a joint as indicators of a joint state. Compared to the first and second embodiment, the third embodiment is different in terms of calculating third and fourth acoustic information. Since step 902 to step 906 are similar to step 102 to step 106 in the first embodiment, the description thereof is omitted, and step 908 and step 910, which are steps different from the first and second embodiments, are described.

In step 908, a basic threshold value and three additional threshold values are set based on the time trend data to calculate the first to fourth acoustic information.

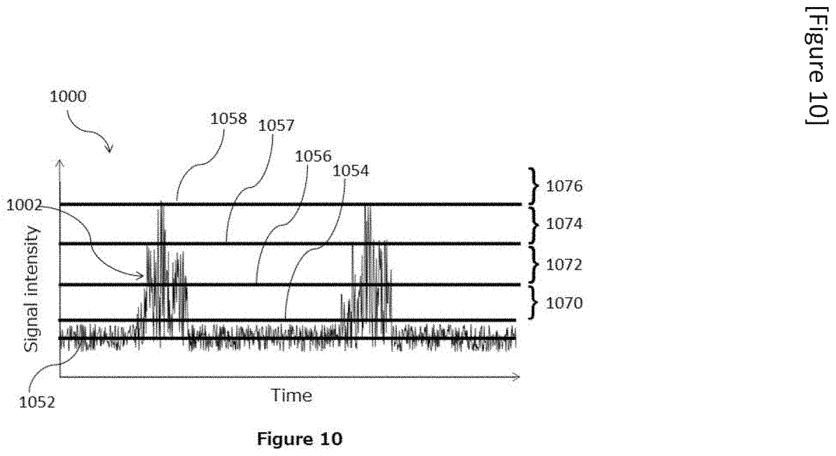

FIG. 10 is a data processing graph 1000 showing a method of setting a basic threshold value 1054, a first additional threshold value 1056, a second additional threshold value 1057 and a third additional threshold value 1058. The graph 1000 includes the first additional threshold value 1056, the second additional threshold value 1057 and the third additional threshold value 1058 in addition to the basic threshold value 1054 which is similar to the basic threshold value 454 in the graph 400 shown in FIG. 4. The basic threshold value 1054 and the first to third additional threshold values 1056, 1057 and 1058 are set to acoustic signal intensities that are different from one another. Each of these additional threshold values are determined by a method similar to those described regarding the first embodiment. In the graph 1000, the third additional threshold value 1058, the second additional threshold value 1057, the first additional threshold value 1056 and the basic threshold value 1054 are set to signal intensities from high to low in that order, wherein, for example, each count of signals having an intensity exceeding each threshold value is calculated as acoustic information. For example, the first additional threshold value 1056 may be a value resulted by adding the product of the standard deviation of the acoustic signal intensity and the second coefficient to the average value 1052 of non-signal intensity, the second additional threshold value 1057 may be a value resulted by adding the product of the standard deviation of the acoustic signal intensity and the third coefficient to the average value 1052 of non-signal intensity, and the third additional threshold value 1058 may be a value resulted by adding the product of the standard deviation of the acoustic signal intensity and the fourth coefficient to the average value 1052 of non-signal intensity. By using a plurality of additional threshold values, each signal is classified into a first signal intensity band 1070 that is the basic threshold value 1054 or higher and below the first additional threshold value 1056, a second signal intensity band 1072 that is the first additional threshold value 1056 or higher and less than the second additional threshold value 1057, a third signal intensity band 1074 that is the second additional threshold value 1057 or higher and less than the third additional threshold value 1058 and a fourth signal intensity band 1076 which is the third additional threshold value 1058 or higher. For example, the first to fourth acoustic information considering both the count of signals and the signal intensity may be calculated by calculating the count of signals of each signal intensity band.

In step 910, a joint state is detected using a combination of the first to fourth acoustic information as an indicator. In one embodiment, when a measurement result is within the range of the first signal intensity band 1070, a joint state is detected as G1, when a measurement result is within the range of the second signal intensity band 1072, a joint state is detected as G2, when a measurement result is within the range of the third signal intensity band 1074, a joint state is detected as G3, and when a measurement result is within the range of the fourth signal intensity band 1076, a joint state is detected as G4. This enables detection of a joint state (e.g., progression phase of knee osteoarthritis) from the first to fourth acoustic information.

In addition, detection of a joint state with higher precision may be carried out by weighting each count of signals of the first to fourth acoustic information (e.g., weighting the count of signals of the first acoustic information to about 1-fold, weighting the count of signals of the second acoustic information to about 2-fold, weighting the count of signals of the third acoustic information to about 3-fold, weighting the count of signals of the fourth acoustic information to about 5-fold). In addition, for example, symptom classification with a discrimination threshold value may be performed using a value resulted by adding up the count of signals of each signal intensity band that was weighted as an indicator.

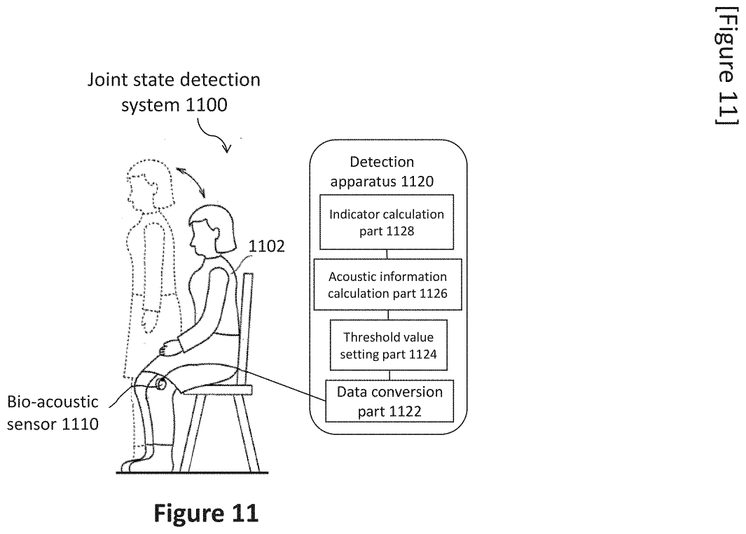

The present application further provides a system of detecting a joint state in accordance with the method of the above-described first, second, or third embodiment. FIG. 11 shows one example of a joint state detection system. The present system comprises a bio-acoustic sensor 1110 obtaining acoustic information from a joint and a detection apparatus 1120 detecting a joint state from a result of acoustic information obtained by the bio-acoustic sensor. The detection apparatus 1120 is connected to the bio-acoustic sensor 1110 with a wire or without a wire. The detection apparatus 1120 may be any computer that can perform the method of the present invention. The detection apparatus 1120 comprises a data conversion part 1122, a threshold value setting part 1124, an acoustic information calculation part 1126 and an indicator calculation part 1128. Each constituent element may be in any form. For example, each constituent element may be a software practiced by a computer, or each may be a separate hardware.

The data conversion part 1122 converts an acoustic signal obtained with a bio-acoustic sensor 1110 to time trend data at least showing the relationship between acoustic signal intensity and time. The data conversion is carried out with the method described above. The data conversion part 1122 sends the converted time trend data to the threshold value setting part 1124.

The threshold value setting part 1124 sets a basic threshold value regarding the acoustic signal intensity from the time trend data received from the data conversion part 1124. The threshold value is set with the method in the first, second, or third embodiment. The threshold value setting part 1124 sends the time trend data and the set threshold value to the acoustic information calculation part 1126.

The acoustic information calculation part 1126 calculates acoustic information based on the threshold value. A method of calculating acoustic information is set with the method in the first, second, or third embodiment. The acoustic information calculation part 1126 sends the calculated acoustic information to the indicator calculation part 1128.

The indicator calculation part 1128 calculates the indicator of a joint state based on the calculated acoustic information. The method for calculating the indicator is calculated with the method in the first, second, or third embodiment. The calculated indicator may be displayed on any display device, transmitted by audio with any speaker device, or stored in any database or the like, as an indicator showing the joint state of a subject.

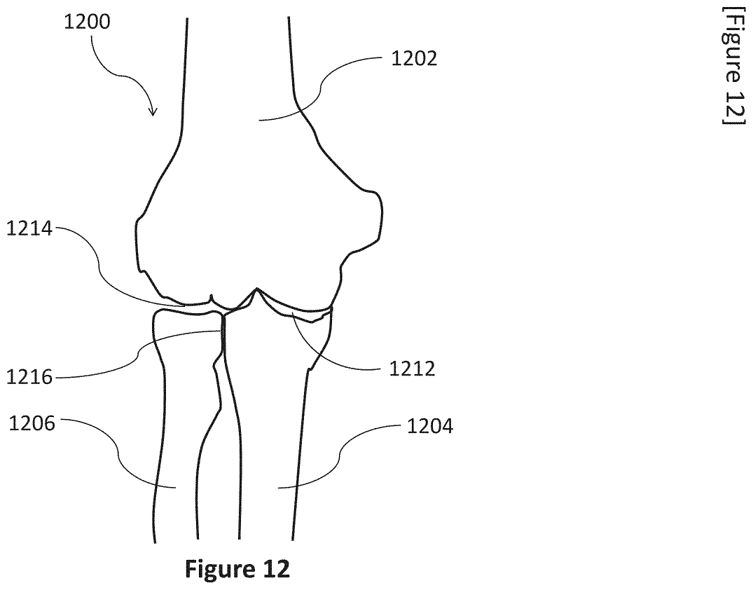

Although the present embodiment described a method and system using acoustic information obtained from a knee joint as an indicator of a joint state, the present method may be applied not only to a knee joint but also to other joints such as an elbow joint, a finger joint, a shoulder joint, a wrist joint, an ankle joint, a hip joint and a jaw joint. For example, FIG. 12 shows a structure 1200 of an elbow joint. The elbow joint is defined among a humerus 1202, an ulna 1204 and a radius 1206. The elbow joint has three joint surfaces, which are a humeroulnar joint 1212, a humeroradial joint 1214 and a radioulnar joint 1216, wherein a cartilage is attached to each joint surface. Thus, it is possible to obtain an acoustic signal caused by the sliding of these cartilages with respect to one another to generate the acoustic information to use as an indicator of a joint state. For example, an acoustic signal may be obtained by wearing an acoustic sensor on a skin near the humeroulnar joint 1212, the humeroradial joint 1214 and/or the radioulnar joint 1216. Acoustic information may be extracted from the obtained acoustic signal with a method similar to the method discussed above.

### EXAMPLES

### Example 1

A test of detecting a joint state of a plurality of subjects was carried out based on the method of the present invention.

A subject carried out a plurality of repeating motion of a sitting position and a standing position using a chair, wherein a joint sound made by a knee joint during this motion was obtained by a bio-acoustic sensor. One bio-acoustic sensor was attached to the patella of the subject, wherein an angle sensor, weighing sensor, and other sensors were not used.

Regarding the acoustic signal obtained by a bio-acoustic sensor as an analog electric signal, the sampling frequency of digital conversion was set to 100 Hz, and the obtained digital signal was converted to time trend data using short-time Fourier transform.

Next, a non-signal time period in the time trend data was identified. The initial time and the terminal time of the non-signal time period were identified under the condition of the absolute value of the average change rate of acoustic signal intensity during 0.01 second being at or below a predetermined value successively for 0.2 seconds. Next, a basic threshold value for excluding a non-signal from the time trend data was identified. The basic threshold value was set to a value resulted by adding 3-fold (first coefficient) of the standard deviation of the intensity of the non-signal to the average value of intensity of the non-signal in the non-signal time period. In addition, a count of acoustic signals with an intensity exceeding the basic threshold value was calculated as acoustic information.

The progression phase of knee osteoarthritis was able to be estimated with the count of acoustic signals.

### Example 2

A test of detecting a joint state of a plurality of subjects was carried out based on the method of the present invention setting a basic threshold value and an additional threshold value and using acoustic information obtained from a joint as an indicator of a joint state.

An acoustic signal was obtained from a subject with a method similar to Example 1 and converted to time trend data of a digital signal.

A non-signal time period was identified and a basic threshold value was set using a method similar to Example 1. Herein, an additional threshold value was further set in addition to the basic threshold value. The additional threshold value was set by multiplying the standard deviation of a non-signal in the non-signal time period by a value that is 6-fold or 9-fold (second coefficient) with a method similar to the method for the basic threshold value. In addition, a count of acoustic signals with an intensity exceeding the threshold value and a count of acoustic signals with an intensity exceeding the additional threshold value were calculated as acoustic information.

The progression phase of knee osteoarthritis was estimated by combining two acoustic information. In addition to the estimation in Example 1, the estimation method herein can identify a subject that possibly has a particularly severe symptom and a subject having the initial symptom of knee arthrosis.

As discussed above, the use of a method using acoustic information obtained from a joint as an indicator of a joint state enabled good detection of a joint state of a subject only with the acoustic information obtained from a bio-acoustic sensor without using a complicated apparatus such as an angle sensor and a weighing meter.

Although the present invention has been exemplified using a preferable embodiment of the present invention as described above, the interpretation of the present invention should not be limited to this embodiment. It is understood that the scope of the present invention should be interpreted by the Claims alone. It is understood that those skilled in the art can practice an equivalent scope based on the description of the present invention and common general knowledge from the description of the specific and preferable embodiment of the present invention. It is also understood that any reference cited herein should be incorporated herein by reference in the same manner as the contents are specifically described herein.

### INDUSTRIAL APPLICABILITY

The present invention is useful as an invention that can provide a method and system of appropriately detecting a joint state from an acoustic signal of a joint which can be easily practiced at a home or the like using only a bio-acoustic sensor.

1. A method of using acoustic information obtained from a joint as an indicator of a joint state, the method comprising:
the step of, using a bio-acoustic sensor, obtaining an acoustic signal emitted by the joint during a time period at least including a first motion period in which the joint changes from a first form to a second form, a pause period of the joint and a second motion period in which the joint changes from the second form to the first form;
the step of converting the acoustic signal that was obtained to time trend data at least showing a relationship between an acoustic signal intensity and time; and
the step of setting a basic threshold value regarding the acoustic signal intensity from the time trend data and calculating first acoustic information based on the basic threshold value,
characterized in that the first acoustic information is used as an indicator of the joint state.

2. The method of claim 1, wherein setting a basic threshold value regarding the acoustic signal intensity from the time trend data further comprises identifying an initial time and a terminal time of at least one non-signal time period from the time trend data and calculating the basic threshold value from time trend data in the non-signal time period.

3. The method of claim 2, wherein at least one of the initial time and the terminal time of the non-signal time period is identified under the condition of an absolute value of an average change rate of the acoustic signal intensity of each micro time being a predetermined value or lower successively throughout a predetermined time period.

4. The method of claim 2, wherein the basic threshold value is calculated based on an average value and a standard deviation of an acoustic signal intensity in the non-signal time period in the time trend data and a first coefficient.

5. The method of claim 1, wherein calculating the first acoustic information based on the basic threshold value comprise calculating the first acoustic information by extracting an acoustic signal intensity exceeding the basic threshold value in the time trend data.

6. The method of claim 1, wherein converting the acoustic signal that was obtained to the time trend data at least showing a relationship between the acoustic signal intensity and time comprises digitally converting the acoustic signal that was obtained on a predetermined sampling frequency.

7. The method of claim 6, wherein the predetermined sampling frequency is about 25 Hz to about 2000 Hz.

8. The method of claim 6, wherein the first acoustic information is a count and/or intensity of the digitally converted acoustic signal exceeding the basic threshold value.

9. The method of claim 1, wherein the first acoustic information is calculated from an acoustic intensity corresponding to the first motion period in the time trend data.

10. The method of claim 1, characterized in that comparison between the first acoustic information and a pre-set discrimination threshold value is used as an indicator of the joint state.

11. The method of claim 1, wherein the step of calculating the acoustic information further comprises setting an additional threshold value of a signal intensity that is different from the basic threshold value and calculating second acoustic information, characterized in that a combination of the first acoustic signal and the second acoustic signal is used as an indicator of the joint state.

12. The method of claim 11, characterized in that comparison between a combination of the first acoustic information and the second acoustic information and a pre-set discrimination value is used as an indicator of the joint state.

13. The method of claim 1, wherein the joint is a knee joint or an elbow joint.

14. A system of detecting a joint state in accordance with the method of claim 1,
the system comprising:
a bio-acoustic sensor obtaining acoustic information from a joint; and
a detection apparatus detecting a joint state from a result of the acoustic information obtained by the bio-acoustic sensor.

15. The system of claim 14, wherein the detection apparatus comprises:
a data conversion part converting the acoustic signal obtained with the bio-acoustic sensor to time trend data at least showing a relationship between an acoustic signal intensity and time;
a threshold setting part setting a basic threshold value regarding the acoustic signal intensity from the time trend data;
an acoustic information calculation part calculating first acoustic information based on the basic threshold value; and
an indicator calculation part calculating an indicator of a joint state based on the first acoustic information that was calculated.

16. The system of claim 14, wherein the system does not comprise an angle sensor.

17. The method of claim 2, wherein identifying an initial time and a terminal time of at least one non-signal time period from the time trend data is carried out without an angle sensor.
